(19) United States
(12) Patent Application Publication
(10) Pub. No.: US 2022/0292534 A1
(43) Pub. Date: Sep. 15, 2022

(54) COMPREHENSIVE SYSTEM OF OPTIMIZING HABIT ENGAGEMENT

(71) Applicant: BJ Fogg, LLC, Healdsburg, CA (US)

(72) Inventors: Brian J. Fogg, Healdsburg, CA (US); Steven Michael Crane, Los Gatos, CA (US)

(21) Appl. No.: 17/202,049

(22) Filed: Mar. 15, 2021

(51) Int. Cl. G06Q 30/02; G16H 20/10; G06Q 10/06

(52) U.S. Cl. CPC G06Q 30/0205; G16H 20/10; G06Q 10/06315

(57) ABSTRACT

Optimizing habit engagement is disclosed, including: obtaining events related to a set of subjects; determining performance segment information corresponding to respective ones of a plurality of habit engagement states based at least in part on the events; and outputting at least a subset of the performance segment information corresponding to the respective ones of the plurality of habit engagement states.

### BACKGROUND OF THE INVENTION

Many organizations want customers or patients to create habits (e.g., of taking medications or using a mobile app daily). The insight those organizations have about their customer/patient habits is limited to purchase data, survey responses, and limited analytics. The existing systems that convey habit data and insights do not represent how habits form over time or how habits lapse. As such, organizations have limited insight about the success and challenges their customers/patients face at different stages of the habit formation process. In addition, because of the limited insight about the habit status of individual (or populations), organizations are not able to design interventions to optimize success in forming, as well as maintaining, a habit. As a result, products and services are not being optimized for engagement (enduring habits), and the organization is less successful because the patient/customer is less successful.

### BRIEF DESCRIPTION OF THE DRAWINGS

Various embodiments of the invention are disclosed in the following detailed description and the accompanying drawings.

FIG. 1 is a diagram showing an embodiment of a system for outputting habit engagement states.

FIG. 2 is a diagram showing an example of a habit engagement states tracking server.

FIG. 3 is a flow diagram showing an embodiment of a process for outputting habit engagement states.

FIG. 4 is a flow diagram showing an example of a process for obtaining configuration information associated with a plurality of habit engagement states.

FIG. 5 is a diagram showing an example schematic illustration of the configuration information associated with a set of four habit engagement states.

FIG. 6 is a flow diagram showing an example of a process for processing a new event associated with a subject.

FIG. 7 is a flow diagram showing an example of a process for updating information related to a plurality of habit engagement states.

FIG. 8 is a flow diagram showing an example of a process for presenting two outputs corresponding to a plurality of habit engagement states.

FIG. 9 is a diagram that shows an example output of a set of habit engagement states pertaining to forming the habit of regularly brushing teeth with an electronic toothbrush.

FIG. 10 is a diagram showing an example user interface for presenting detailed information associated with a set of habit engagement states.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

Embodiments of a system of optimizing habit engagement are described. Events related to a set of subjects are obtained. In various embodiments, an event is recorded by a device that detects the event using one or more sensors or another activity detecting mechanism. In some embodiments, events are determined from surveys (e.g., completed by customers, family members of customers, and/or medical providers), a third-party data feed, and/or industry-wide research. In various embodiments, a “subject” comprises an individual or a set of individuals that are candidates in forming one or more habits. For example, an event describes one or more activities that have been performed by a subject. In various embodiments, a “habit” comprises an acquired mode of behavior by a subject. In various embodiments, the events are obtained (e.g., at periodic time intervals or whenever they became available) over a window of time. Performance segment information corresponding to respective ones of a plurality of habit engagement states is determined based on the obtained events. In various embodiments, a “habit engagement state” comprises a condition/milestone/stage that a subject has reached by having completed one or more activities (e.g., over a predetermined length of time). In various embodiments, each habit engagement state is associated with at least one performance segment. For example, each habit engagement state is associated with both a successful performance segment and an unsuccessful performance segment. Each subject that has been or is currently associated with a state is determined to be included in one of the performance segments corresponding to that state based on the subject's obtained history of events. In some embodiments, the plurality of habit engagement states describes stages that are associated with one or more (e.g., related) target habits. In various embodiments, logic is stored for each habit engagement state to describe which subjects belong to which performance segments corresponding to that habit engagement state. In various embodiments, logic is also stored for the plurality of habit engagement states to describe how/when subjects transition from one habit engagement state to another. As events with respect to various subjects are collected over time, which habit engagement states the subjects have belonged to and/or currently belong to, as well as the performance segments corresponding to which each habit engagement state the subjects belong, are determined. The performance segment information corresponding to each of the plurality of habit engagement states is then output. In some embodiments, the performance segment information corresponding to each of the plurality of habit engagement states is output as visualizations/graphical representations showing the performance segment information for each habit engagement state. In some embodiments, the performance segment information corresponding to each of the plurality of habit engagement states is output as a data feed with the performance segment information for each habit engagement state as well as suggested interventions to be performed with respect to one or more target subsets of subjects (e.g., to encourage those subjects to form a habit).

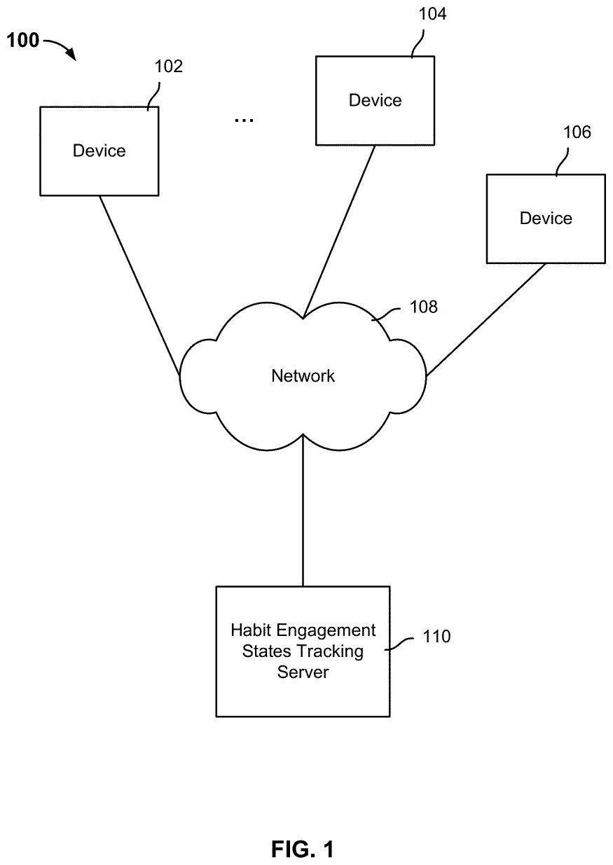

FIG. 1 is a diagram showing an embodiment of a system for outputting habit engagement states. In the example of FIG. 1, system 100 includes device 102, device 104, device 106, network 108, and habit engagement states tracking server 110.

Each of devices 102, 104, and 106 may be a smart appliance, a mobile device, and/or any other type of computing device. Each of devices 102, 104, and 106 is configured to send and receive data over a network such as network 108. In various embodiments, devices, including, but not limited to, devices 102, 104, and 106 are configured to collect events associated with one or more subjects. The events that are collected by devices, including, but not limited to, devices 102, 104, and 106 are sent by the devices over network 108 to habit engagement states tracking server 110. In some embodiments, a device is configured to detect (e.g., via one or more sensors) events that occur with respect to one or more subjects. In some embodiments, a device is configured to receive an input (e.g., survey information, a third-party data feed, and/or industry-wide research) that describes events that have occurred with respect to one or more subjects. In various embodiments, an event comprises identifying information associated with the relevant subject, associated time information, and information describing an activity. For example, a device comprises a network-connected personal appliance (e.g., a toothbrush) that is configured to record when and for how long each time a subject uses the toothbrush to brush his or her teeth. In another example, a device comprises a smart phone that executes an application through which a subject (e.g., a patient for whom a certain medication has been prescribed) or another individual inputs information describing an event (e.g., when and how the patient has taken the medication) that has occurred with respect to the subject.

Habit engagement states tracking server 110 is configured to obtain events associated with one or more subjects from devices (e.g., devices 102, 104, and 106) (and other sources of data) and use the events to generate performance segment information corresponding to a set of two or more habit engagement states. For example, the set of habit engagement states may describe a series of different stages that lead up to and away from the formation of a habit. For example, one habit engagement state in the set may have one or more adjacent habit engagement states to which a subject can transition to, based on obtained events associated with the subject. Habit engagement states tracking server 110 is configured to store configuration information pertaining to the set of two or more habit engagement states. In various embodiments, the configuration information pertaining to the set of two or more habit engagement states comprises at least identifying information associated with each state, transition logic for determining how/when a subject transitions in between adjacent states, and segmenting logic for determining how/when a subject belongs to which performance segment corresponding to each state. Habit engagement states tracking server 110 is configured to determine, for a subject for which new event(s) were received, whether the new event(s) would cause the subject to belong to a different performance segment corresponding to the current habit engagement state to which the subject currently belongs and/or whether the new event(s) would cause the subject to transition to another habit engagement state (e.g., one that is adjacent to the subject's stored current habit engagement state). Habit engagement states tracking server 110 is further configured to determine (e.g., periodically or in response to a user or programmatic instruction), for each habit engagement state, for the number of subjects that are or have been associated with that state, which portion (e.g., percentage) of those subjects belong to each of one or more respective performance segments. In some embodiments, each habit engagement state is associated with at least one performance segment (e.g., a successful performance segment, an unsuccessful performance segment, and/or an in-state performance segment). In some embodiments, the unsuccessful performance segment is divided into two or more subsegments, where each subsegment of the unsuccessful performance identifies a particular portion of subjects in the unsuccessful performance segment that is unsuccessful segment based on a particular common set of attributes (e.g., lacking motivation, lacking ability, and/or lacking both motivation and ability). Habit engagement states tracking server 110 is configured to therefore determine at least performance segment information corresponding to each habit engagement state of the set of two or more habit engagement states at each of various times. Habit engagement states tracking server 110 is configured to output the performance segment information corresponding to each habit engagement state of the set of two or more habit engagement states at each of various times to a device (e.g., the device of a habit dashboard user that is monitoring the habit engagement progress of a set of subjects), at which the outputted data can be presented, saved, opened, and/or transferred, etc. In some embodiments, the output performance segment information may be presented at a user interface (which is sometimes referred to as a dashboard) that shows at least a portion of the set of habit engagement states, where for each habit engagement state, the user interface presents a visualization/representation that denotes the portion of subjects that are or have been associated with that habit engagement state that is associated with a first performance segment and the portion of subjects that are or have been associated with that habit engagement state that is associated with a second performance segment. In some embodiments, the user interface further presents representations of quantities of subjects that have transitioned into and/or out of each habit engagement state relative to an adjacent habit engagement state. In some embodiments, habit engagement states tracking server 110 stores the output performance segment information corresponding to a set of habit engagement states corresponding to each of multiple times such that a habit dashboard user can select to switch between viewing the outputs associated with different times to understand how the subjects' behaviors have changed over time. In some embodiments, habit engagement states tracking server 110 is configured to output performance segment information corresponding to a set of habit engagement states in the form of exporting the data (e.g., as via an application programming interface (API)).

In some embodiments, the configuration pertaining to the set of two or more habit engagement states may be updated (e.g., based on a submission by an administrator) over time. Changing the configuration pertaining to the set of two or more habit engagement states may cause the determination of how and when subjects are associated with each state and which performance segment thereof, to also change. Thereby, the configurability of the configuration pertaining to the set of two or more habit engagement states allows the corresponding determination and output of habit engagement state related performance segment information to remain flexible and dynamic in response to different goals for tracking subject habit engagement behavior.

Various embodiments of outputting habit engagement states described herein enable granular information pertaining to subjects to be collected and used to update performance segment information corresponding to a set of two or more habit engagement states. Due to the habit engagement states representing different steps/stages in the process of forming (and slipping away from) habits, the presentation (“dashboard”) of the habit engagement states with their respective performance segment information as described herein provide an elegant and holistic view of subjects' behavior. Such a presentation can provide a quick summary as to where, if any, in the progress of subjects' behavior towards forming a habit, progress is made and where, if any, intervention is needed to encourage the subjects towards forming or returning to a target habit/state.

As will be described in further detail herein, habit engagement states tracking server 110 enables tracking of a set of subjects as they progress/transition through a set of habit engagement states. Habit engagement states tracking server 110 also provides an output of a presentation of the current snapshot at a corresponding time of the subjects' progress with respect to the habit engagement states, which allows a granular view of how far along the subjects are towards the goals of habit formation. For example, the presentation is a dynamic visualization that includes graphical representation of each habit engagement state. The dynamic visualization can be interactive such that a habit dashboard user can select a portion of the visualization to cause additional (e.g., more detailed) information to be presented. In a specific example, if a habit dashboard user selected a graphical representation related to a particular habit engagement state, then additional information may be presented on the behavior of subjects over time with respect to that state and/or on recommended interventions to encourage subjects in that state to transition to an adjacent state.

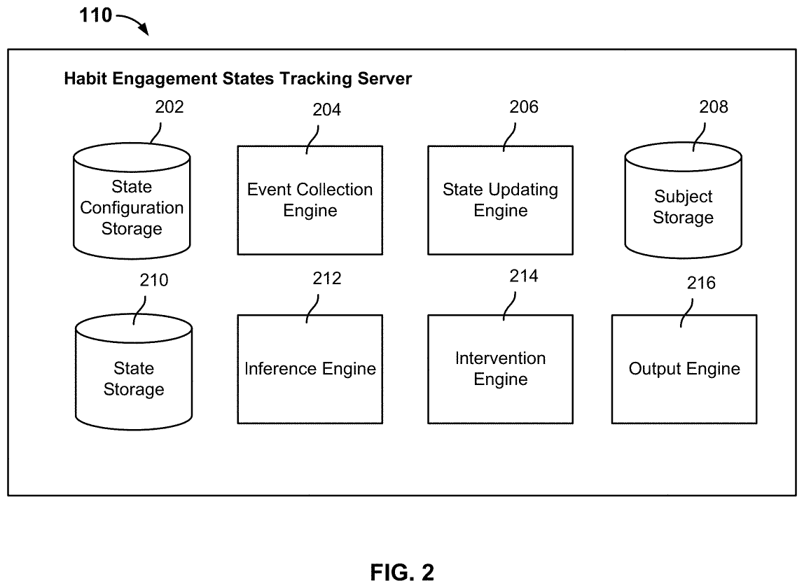

FIG. 2 is a diagram showing an example of a habit engagement states tracking server. In some embodiments, habit engagement states tracking server 110 of system 100 may be implemented using the example described in FIG. 2. The example habit engagement states tracking server of FIG. 2 includes state configuration storage 202, event collection engine 204, state updating engine 206, subject storage 208, state storage, 210, inference engine 212, intervention engine 214, and output engine 216. Each of event collection engine 204, state updating engine 206, inference engine 212, intervention engine 214, and output engine 216 can be implemented, for example, as distinct or integrated software components (or specification documents for the building of such software components), which can include module(s), package(s), and/or other distinct or integrated sub-components to provide an executable computer program that can perform these described functions when executed on a processor, and can be implemented using a programming language such as Go, Java, Python, Objective C, and/or other programming languages. An example hardware computing environment to execute the components of FIG. 2 includes a cloud computing service, such as Amazon's Web Services. Each of state configuration storage 202, subject storage 208, and state storage 210 may be implemented as one or more databases (e.g., MySQL).

State configuration storage 202 is configured to store configuration information pertaining to a set of two or more habit engagement states. For a set of habit engagement states, a set of configuration information may include, for example, but is not limited to: identifying information associated with each state within the set, transition logic for determining whether/how (e.g., which condition(s) are to be met for) a subject to transition from one state to an adjacent state in the set, and segmenting logic for determining one of at least one performance segment to which a subject belongs for each state in the set that he or she has been or is currently associated with. In some embodiments, both the transition logic and the segmenting logic associated with a set of habit engagement states describe conditions/criteria associated with a subject's set of historical events, which describe activities performed by the subject and when the activities were performed. For example, a set of habit engagement states may describe the formation of one or more (e.g., related) habits. In some embodiments, the set of configuration information associated with a set of habit engagement states may be updated over time (e.g., in response to new administrator input(s) via a user interface for updating configuration information) such that the conditions in which subjects are determined to transition between states and/or among different performance segments within a state can remain flexible. For example, the transition logic for determining that a subject transitions to (e.g., becomes associated with) State A may be that a subject such as Subject X needs to have completed a predetermined activity. In another example, after Subject X has been determined to be associated with State A, the segmenting logic for determining whether Subject X belongs to either the successful performance segment of State A or the unsuccessful performance segment of State A is based on whether Subject X has completed a specified number of instances of the predetermined activity within a predetermined period of time.

Event collection engine 204 is configured to obtain events associated with subjects. In various embodiments, event collection engine 204 is configured to receive events from survey data, industry-wide research, a third-party data feed, or devices that include sensor(s) that are configured to detect activity performed by a subject. For example, a device that includes sensor(s) that are configured to detect activity performed by a subject is a network-connected electronic toothbrush that detects when a subject is brushing his or her teeth. As such, the network-connected electronic toothbrush can detect whether a target toothbrushing activity has occurred and then send the detected activity as an event to the habit engagement states tracking server. In some embodiments, event collection engine 204 is configured to receive events from devices that execute a web browser or other application that provides a user interface through which a subject can input events. For example, a device that executes a web browser or other application that provides a user interface through which a subject can input events is a smart phone. In some embodiments, event collection engine 204 is configured to query devices for new events. In some embodiments, event collection engine 204 is configured to receive events from devices without initially querying the devices. In various embodiments, an event comprises identifying information associated with the relevant subject, associated time information, and information describing an activity. In some embodiments, events that are collected by event collection engine 204 are sent to state updating engine 206 for state updating engine 206 to process.

State updating engine 206 is configured to process events and use the events to update stored information related to subjects. In various embodiments, for a new event, state updating engine 206 is configured to determine the relevant subject associated with the new event. Then, state updating engine 206 is configured to determine the habit engagement state of a set to which the subject currently belongs based on information related to the subject that is stored at subject storage 208. For the habit engagement state to which the subject currently belongs, state updating engine 206 is configured to determine to which performance segment the subject belongs based at least in part on the new event, the set of historical events associated with the subject, and the segmenting logic associated with the habit engagement state to which the subject currently belongs. For example, after determining the performance segment to which the subject belongs, state updating engine 206 is configured to update data stored at either or both of subject storage 208 or state storage 210 that indicate the subject's membership to the determined performance segment for the subject's currently associated habit engagement state. Furthermore, state updating engine 206 is configured to determine whether the subject is to transition to another habit engagement state that is adjacent to the state with which the subject is currently associated based at least in part on the new event, the set of historical events associated with the subject, and the transition logic associated with the habit engagement state to which the subject currently belongs and an adjacent state. For example, if state updating engine 206 determines that the subject has transitioned to be associated with another habit engagement state, state updating engine 206 is configured to update data stored at either or both of subject storage 208 or state storage 210 that indicates the subject's membership with the new habit engagement state. In some embodiments, in response to determining that a subject has transitioned into a predetermined habit engagement state, state updating engine 206 is configured to programmatically send an alert, a message, and/or an intervention to the subject to encourage them to perform one or more specified activities. For example, the predetermined habit engagement state that may be associated with such a sending of an alert, a message, and/or an intervention may be a state that is moving away from forming a habit and is therefore an undesirable state. In some embodiments, output engine 216 is configured to determine a habit engagement score corresponding to a particular subject based at least in part on that subject's previously and currently associated habit engagement states and/or the subject's previously and/or currently associated performance segments within those states. A subject's habit engagement score may represent the subject's likelihood to form and/or maintain a habit.

State updating engine 206 is further configured to update performance segment information for each habit engagement state in a set of habit engagement states based on the updated subject information. In some embodiments, state updating engine 206 is configured to update performance segment information for each habit engagement state on a periodic basis. In some embodiments, state updating engine 206 is configured to update performance segment information for each habit engagement state in response to a user instruction (e.g., from a habit dashboard user). In some embodiments, state updating engine 206 determines for each habit engagement state, a respective portion of subjects, which have been or are currently associated with the state that is included in each different performance segment, that is associated with that state. For example, state updating engine 206 may determine the respective percentage of subjects, which have been or are currently associated with the state that is included in each different performance segment, that is associated with that state. State updating engine 206 is configured to store the determined performance segment information for each habit engagement state in state storage 210. In some embodiments, state updating engine 206 may receive one or more filters comprising criteria for types of subjects for which performance segment information is to be determined for each habit engagement state in a set. For example, a habit dashboard user may select filters for receiving performance segment information for subjects that are male and between 18 to 35 years old. In response to the selected filter(s), state updating engine 206 is configured to identify the subset of subjects that match the criteria and then determine performance segment information for each habit engagement state (e.g., the determination of the percentage of subjects in each performance segment of each state) based on that identified subset of subjects.

Inference engine 212 is configured to determine an inference associated with a subject based on the subject's collected events and/or reached habit engagement states so far and on the analysis of the performance segment information related to the set of habit engagement states. In some embodiments, inference engine 212 is configured to derive one or more probabilities of how likely a subject is to transition from one habit engagement state to a later habit engagement state given a set of events and/or currently/previously associated with habit engagement states that have been determined for the subject. Inference engine 212 is configured to derive such probabilities through analyzing the performance segment information for the set of habit engagement states at different points in time. In some embodiments, a machine learning model can be trained on the performance segment information of a set of subjects for the set of habit engagement states and the determined probabilities that a subject at one given state would transition (e.g., within a certain period of time) to a later state. Inference engine 212 can then input a subject's current habit engagement state information (e.g., the subject's collected events so far, the subject's current performance segment associated with the subject's current habit engagement state) into the machine learning model to receive the model's output of the probability that the subject would transition to a later habit engagement state in the set of habit engagement states. For example, inference engine 212 is configured to determine that a subject that reaches State 1 immediately and then also transitions to States 2 and 3 quickly are very likely to reach State 4 and stay there, whereas another subject who lag in reaching States 1 and 2 is not likely to reach State 4. Put another way, inference engine 212 is configured to determine, for a subject, an inference that comprises a probability that the subject will reach a later habit engagement state, given the subject's current habit engagement state information. In a first example, the inference that is determined by inference engine 212 for a subject can be used to determine whether an intervention is needed to be provided to the subject (e.g., to encourage the subject to perform the activities that will lead the subject to transition to a later habit engagement state to which the inference indicates that there is currently a low probability that the subject will transition). In a second example, the inference that is determined by inference engine 212 for a subject can be used to associate a predetermined status with the subject. The predetermined status with the subject can then be indicated to a third-party and/or used to determine whether an action (e.g., an increase of insurance premium) should be performed with respect to the subject.

Intervention engine 314 is configured to provide an intervention to a subject. For example, an intervention can be a customized message (e.g., a text message, an email), a prompt (e.g., a pop window), a video (e.g., that shows a step-by-step guide to performing one or more activities), and/or another type of dynamically generated media. In various embodiments, intervention engine 314 is configured to programmatically design a customized intervention that will help/encourage a target subject to perform one or more specified activities. In some embodiments, intervention engine 314 is configured to provide an intervention to a subject in response to one or more triggers. Examples of such triggers may include a determination that the subject has transitioned to a predetermined habit engagement state (e.g., as a result of the subject's absence of performing an activity within a given period of time) and/or an inference determined for the subject that indicates a lower than a predetermined probability that the subject will advance to a later habit engagement state. In some embodiments, intervention engine 314 is configured to select a pre-generated intervention and then to customize the selected intervention for a subject based on one or more of the following: the trigger to send the intervention, the subject's current habit engagement state, the subject's current performance segment corresponding to the subject's current habit engagement state, and/or the set of historical events that have been obtained for the subject.

Output engine 216 is configured to output performance segment information corresponding to each habit engagement state in a set of habit engagement states. In various embodiments, output engine 216 is configured to output performance segment information corresponding to each habit engagement state in a set of habit engagement states by presenting a user interface (e.g., by sending data that is usable to render the user interface to a device) that shows, for each habit engagement state, a corresponding visualization that shows a corresponding portion of subjects that belong to each performance segment associated with that state. In some embodiments, the user interface also shows representation associated with the number, percentage, or portion of subjects that transition between adjacent habit engagement states. In some embodiments, each output of performance segment information corresponding to each habit engagement state in a set of habit engagement states is associated with a corresponding time information and therefore, output engine 216 may cache outputs of performance segment information corresponding to each habit engagement state in the set of habit engagement states at different times. The cached performance segment information corresponding to each habit engagement state associated with different time information may be presented at the user interface to illustrate (in an animation, for example) how the subjects' progress through the habit engagement states have changed over time.

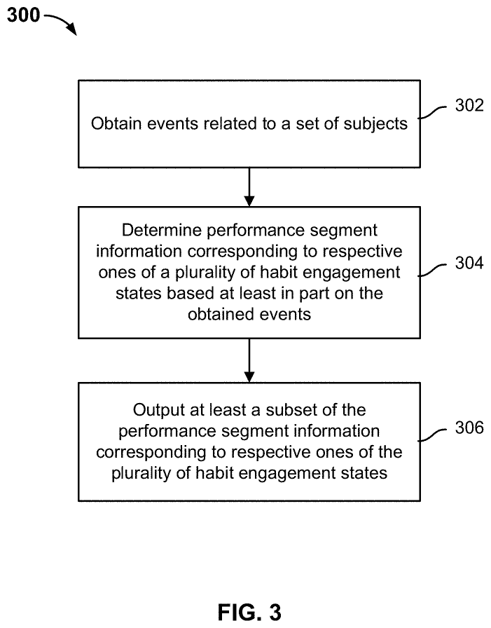

FIG. 3 is a flow diagram showing an embodiment of a process for outputting habit engagement states. In some embodiments, process 300 is implemented by habit engagement states tracking server 110 of system 100 of FIG. 1.

At 302, events related to a set of subjects are obtained. In various embodiments, events related to a set of subjects are received from one or more devices, surveys, a third-party data feed, and/or industry-wide research. For example, a device is a specialized device that includes one or more sensors that are configured to detect, record, and report subject activity. In another example, a device is a mobile device that is configured to receive user input that describe subject activity. In another example, a web browser plug-in is configured to track subject events on a journaling website. As mentioned above, in various embodiments, an event comprises identifying information associated with the relevant subject, associated time information, and information describing an activity or absence of an activity. As mentioned above, in various embodiments, a subject comprises an individual (e.g., a customer, a patient, or a volunteer) or a set of individuals that are candidates in forming one or more habits.

At 304, performance segment information corresponding to respective ones of a plurality of habit engagement states is determined based at least in part on the obtained events. The obtained events as well as configuration information associated with the plurality of habit engagement states are used to determine to which habit engagement states the subjects have previously belonged and/or currently belong. Then, based on the habit engagement states to which the subjects have previously belonged and/or currently belong, in various embodiments, the respective percentage breakdown of subjects that are currently and/or have been previously associated with each habit engagement state corresponding to each performance segment of the state is determined as part of the performance segment information corresponding to the state. In some embodiments, in addition to a representation associated with the number, percentage, direction, inferences of future states, and/or portion of subjects that have transitioned from one habit engagement state to another, an adjacent habit engagement state is also determined.

At 306, at least a subset of the performance segment information corresponding to respective ones of the plurality of habit engagement states is outputted. In various embodiments, a presentation of the performance segment information corresponding to at least some of the habit engagement states of the plurality of habit engagement states is caused to be presented at a user interface. In some embodiments, the output presentation comprises a dynamic visualization. For example, the dynamic visualization shows for each of at least a subset of the habit engagement states, a corresponding performance segment information. Furthermore, portions of the dynamic visualization can be selected to cause additional (e.g., more detailed) information on the selected portion to be presented. For example, the visualization shows for at least some of the habit engagement states that are adjacent to each other (e.g., states for which subjects can transition from one directly to the other), the representation of the number, percentage, direction, and/or portion of subjects that have transitioned between the adjacent states. In some embodiments, the output presentation comprises text, statistics, and/or images that summarize the segment information corresponding to at least some of the habit engagement states of the plurality of habit engagement states. In some embodiments, the output of the performance segment information corresponding to respective ones of the plurality of habit engagement states is a data export.

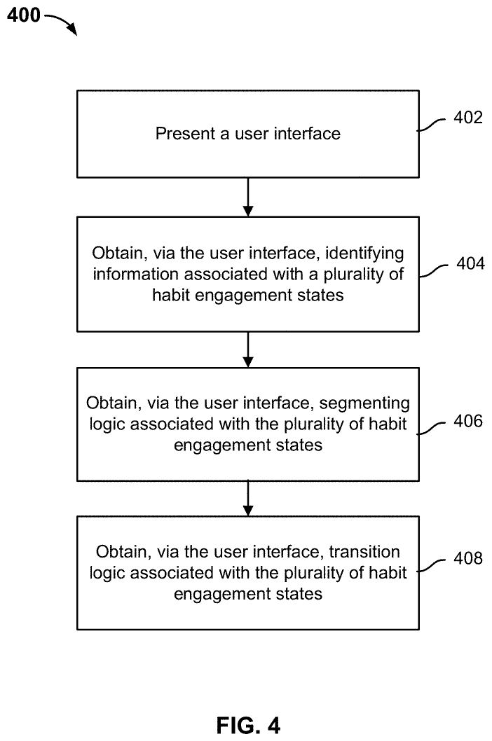

FIG. 4 is a flow diagram showing an example of a process for obtaining configuration information associated with a plurality of habit engagement states. In some embodiments, process 400 is implemented by habit engagement states tracking server 110 of system 100 of FIG. 1.

At 402, a user interface is presented. For example, the user interface is caused to be presented (e.g., via a web browser or by execution of an application) at a device that is used by an administrator user to configure a set of habit engagement states. As described herein, the performance segment information is maintained for the set of habit engagement states for a habit dashboard user to monitor the progress of subjects through the set of habit engagement states.

At 404, identifying information associated with a plurality of habit engagement states is obtained via the user interface. For example, identifying information associated with the habit engagement states describes the names of the states, which one or more performance segments are associated with each state, and/or which states are adjacent to each other (such that a subject can directly transition from one state to its adjacent state). In some embodiments, each habit engagement state may include at least one performance segment.

At 406, segmenting logic associated with the plurality of habit engagement states is obtained via the user interface. For example, segmenting logic describes the conditions/criteria associated with classifying a subject into each respective performance segment of a habit engagement state. For example, the conditions/criteria described in the segmenting logic is related to activities that a subject has performed over a specified period of time. In a specific example, the segmenting logic specifies computing the information associated with a particular performance segment of a particular habit engagement state as a function of a specified particular numerator and a specified denominator.

At 408, transition logic associated with the plurality of habit engagement states is obtained via the user interface. For example, transition logic describes the conditions/criteria associated with classifying a subject that has not been associated with any state into a particular state. For example, transition logic describes the conditions/criteria associated with classifying a subject that is currently associated with a first habit engagement state to be associated with a second habit engagement state, where the second habit engagement state is adjacent to the first habit engagement state in the set of habit engagement states. For example, the conditions/criteria described in the segmenting logic are related to activities that a subject has performed over a specified period of time.

In some embodiments, the configuration information associated with a set of habit engagement states may also include conditions/criteria for when a reminder message, prompt, and/or other targeted intervention should be sent to a device associated with a subject. For example, the conditions/criteria may be associated with the subject being determined to have transitioned to a less desirable habit engagement state (e.g., a state in which the subject is failing to form the target habit), after which the subject could be prompted to perform one or more activities in order to transition out of that state.

The configuration information associated with a set of habit engagement states may be updated over time by the administrator. For example, updating configuration information associated with a set of habit engagement states includes updating the number of habit engagement states in the set, the conditions/criteria included in the segmenting logic, and the conditions/criteria included in the transition logic. Updating the configuration information associated with a set of habit engagement states would affect the determination of how subjects progress through the habit engagement states and would subsequently affect the output of performance segment information related to the set of habit engagement states.

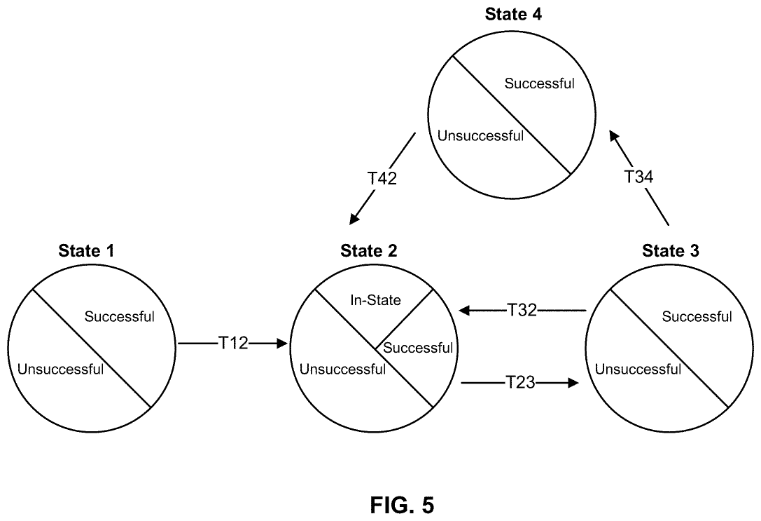

FIG. 5 is a diagram showing an example schematic illustration of the configuration information associated with a set of four habit engagement states. In the example of FIG. 5, the set of habit engagement states includes State 1, State 2, State 3, and State 4. Each of the four habit engagement states includes respective two or more performance segments to which subjects that currently or have previously been associated with the state can belong. Habit engagement States 1, 3, and 4 each includes two performance segments: a successful performance segment and an unsuccessful performance segment. Habit engagement State 2 includes three performance segments: a successful performance segment, an in-state performance segment, and an unsuccessful performance segment. The configuration information associated with the set of four habit engagement states includes segmenting logic that describes for each of States 1, 2, 3, and 4, conditions/criteria for subjects to belong to the successful performance segment or the unsuccessful performance segment of each state. Adjacent states in the set of four habit engagement states are states to which a subject that currently belongs to one state can transition to, in accordance to the transition logic. In the example of FIG. 5, State 2 is an adjacent state to State 1 because a subject currently associated with State 1 can transition to State 2 by meeting transition criteria T12. State 2 and State 3 are adjacent states to each other because a subject currently associated with State 2 can transition to State 3 by meeting transition criteria T23 or a subject currently associated with State 3 can transition to State 2 by meeting transition criteria T32. State 4 is an adjacent state to State 3 because a subject currently associated with State 3 can transition to State 4 by meeting transition criteria T34. State 2 is an adjacent state to State 4 because a subject currently associated with State 4 can transition to State 2 by meeting transition criteria T42.

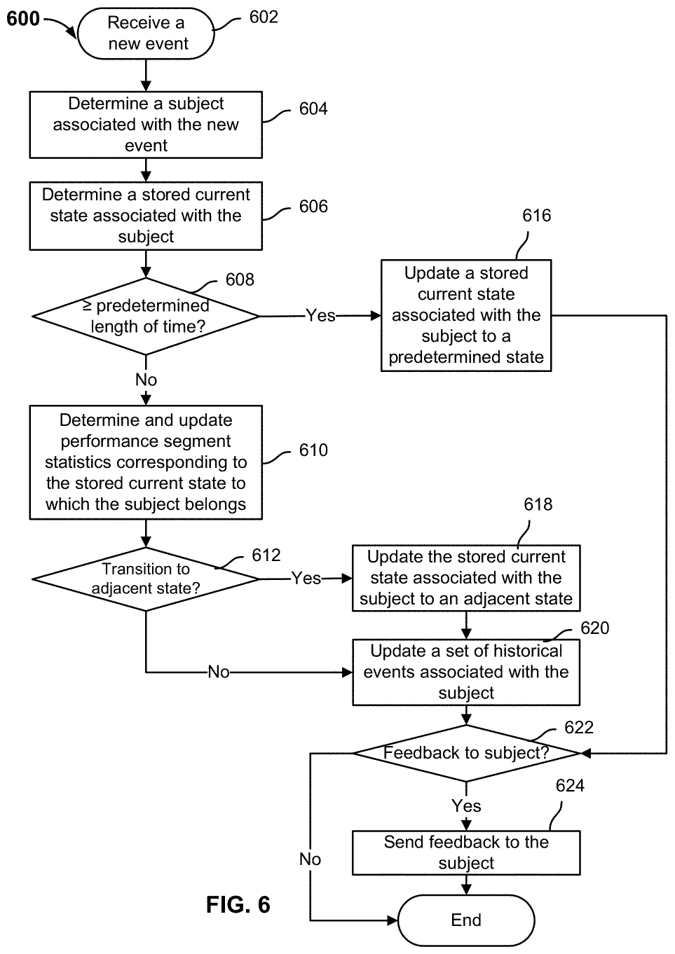

FIG. 6 is a flow diagram showing an example of a process for processing a new event associated with a subject. In some embodiments, process 600 is implemented by habit engagement states tracking server 110 of system 100 of FIG. 1. In some embodiments, step 302 of process 300 of FIG. 3 is implemented, at least in part, using process 600.

At 602, a new event is received. For example, the new event is received from a device. The event includes identifying information associated with the relevant subject, one or more activities that have been performed by the subject, and time information associated with the occurrence of the one or more activities. In some embodiments, source-identifying information associated with the source from which the new event was obtained is also received. For example, the source-identifying information may indicate that the event was recorded by a specified device or obtained through subject or user input (e.g., as part of a survey).

At 604, a subject associated with the new event is determined.

At 606, a stored current habit engagement state associated with the subject is determined and stored. In some embodiments, the current habit engagement state associated with each subject is stored along with each previous habit engagement state with which the subject was associated.

At 608, whether at least a predetermined length of time has elapsed since a previous event associated with the subject was received is determined. In the event that at least a predetermined length of time has elapsed since a previous event associated with the subject was received is determined, control is transferred to 616. Otherwise, in the event that a predetermined length of time has not elapsed since a previous event associated with the subject was received is determined, control is transferred to 610. If this new event has been received after the predetermined length of time has elapsed since a previous event associated with the subject was received, then it is determined that the subject transitions to a predetermined state. For example, if the update (e.g., new event) associated with the subject had been received after a long period (e.g., over the predetermined of time) since the last event associated with the subject had been received, then the long period of inactivity (as represented by the longer than the predetermined length of time between when the new event was received relative to when the previous event had been received) indicates that the subject should transition into a predetermined habit engagement state (as described in step 616, below), which could be associated with a state that indicates lapsing from forming a habit.

At 616, a stored current habit engagement state associated with the subject is updated to a predetermined habit engagement state. For example, if the new event has been received after the predetermined length of time has elapsed since a previous event associated with the subject was received, then regardless of the current habit engagement state with which the subject is associated, the subject is to transition to a predetermined habit engagement state (e.g., a state that is associated with a period of inactivity and is indicative that a habit has not been successfully formed).

At 610, performance segment statistics corresponding to the stored current habit engagement state to which the subject belongs is determined and updated. Based at least on the subject's new event, the subject's stored set of historical events, and segmenting logic associated with the subject's stored current habit engagement state, the performance segment of the subject's stored current habit engagement state to which the subject belongs can be determined. Then, in some embodiments, statistics (e.g., the number of subjects that are) associated with the determined performance segment of the subject's stored current habit engagement state are updated (e.g., incremented by one). In some embodiments, performance segment memberships associated with the subject are also determined to indicate that the subject is associated with the determined performance segment of the subject's stored current habit engagement state.

For example, for the subject's stored current habit engagement state, State 1, the segmenting logic indicates that if the subject has completed the target activity 28 or more times in the last 30 days, then the subject is determined to be in the successful performance segment of that habit engagement state. The segmenting logic further indicates that if the subject has completed the target activity more than 21 times but fewer than 24 times in the last 30 days, then the subject is determined to be in the in-state performance segment of that habit engagement state. The segmenting logic also indicates that if the subject completed the target behavior on 20 or fewer days out of the last 30 day, then the subject is determined to be in the unsuccessful performance segment of that habit engagement state. Assume that based on the subject's new event and set of historical events, it is determined that the subject has done the target activity/behavior 23 times within the last 30 days, then the subject is determined to belong to the in-state performance segment of State 1. As a result, the statistics associated with the in-state successful performance segment of State 1 are updated to include an additional subject. Furthermore, the subject's own performance segment memberships are updated to indicate that the subject belongs to the in-state performance segment of State 1.

At 612, it is determined whether the subject should transition to an adjacent habit engagement state. In the event that the subject should transition to an adjacent habit engagement state, control is transferred to 618. Otherwise, in the event that the subject should not transition to an adjacent habit engagement state, control is transferred to 620.

Based at least on the subject's new event, the subject's stored set of historical events, and transition logic associated with the subject's stored current habit engagement state and one or more adjacent habit engagement states, whether the subject should be transitioned from the stored current habit engagement state to an adjacent habit engagement state can be determined. If the subject's activities as described by the new event and the subject's set of historical events meet the conditions/criteria of the transition logic to transition into an adjacent habit engagement state, then the subject is determined to have transitioned into that adjacent habit engagement state. Otherwise, if the subject's activities as described by the new event and the subject's set of historical events does not meet the conditions/criteria of the transition logic to transition into an adjacent habit engagement state, then the subject is determined to remain in his or her stored current habit engagement state. Then, in some embodiments, if it is determined that the subject has transitioned to an adjacent habit engagement state, then statistics (e.g., the number of subjects that are) associated with the adjacent habit engagement state are updated (e.g., incremented by one).

For example, the subject's stored current habit engagement state is State 1 and transition logic between State 1 and its adjacent habit engagement state, State 2, indicates that if the subject has completed the target activity 28 or more times in the last 30 days, then the subject is determined to transition from being associated with State 1 to being associated with State 2. Assume that based on the subject's new event and set of historical events, it is determined that the subject has completed the target activity 28 or more times in the last 30 days, then the subject is determined to transition to State 2. As a result, the statistics associated with the successful performance segment of State 2 are updated to include an additional subject.

At 618, the stored current state associated with the subject is updated to the adjacent habit engagement state. Because the subject's activities that were described by the subject's events met the conditions/criteria for transitioning to the adjacent habit engagement state, the subject is now associated with the adjacent habit engagement state and therefore, the subject's stored current state refers to the adjacent habit engagement state.

At 620, a set of historical events associated with the subject is updated. The stored set of historical events associated with the subject is updated to include the new event.

At 622, whether feedback should be sent to the subject is determined. In the event that feedback should be sent to the subject, control is transferred to 624. Otherwise, in the event that feedback should not be sent to the subject, process 600 ends. In some embodiments, the set of historical events associated with the subject and/or the stored current habit engagement state associated with the subject may meet conditions/criteria for sending feedback to the subject.

At 624, feedback is sent to the subject. In some embodiments, the feedback is in the form of an alert, a prompt, or an intervention. In some embodiments, the feedback is programmatically customized to the subject based on one or more of the following: the set of historical events associated with the subject, the subject being associated with the predetermined habit engagement state (e.g., at determined at step 616), and/or the subject being associated with a particular performance segment in the subject's current habit engagement state. The feedback may be congratulatory (e.g., to celebrate the subject being associated with a state of forming or having formed a habit) or interventional (e.g., to encourage the subject to behave in a way so as to form a habit). In a first example, if a subject has been associated with a particular habit engagement state for a predetermined period of time, it can be determined that an intervention should be sent to the subject to motivate, guide, enable, prompt, and/or encourage the subject to perform a target activity. In some embodiments, a period of time measured from when a subject enters a less desirable state, after which, it is observed that 50% or 20% of subjects do not return to a more desirable state is sometimes called a “drop off point.” The drop off point may be considered as defining a window of opportunity in which some sort of intervention for the subject may nudge the subject towards performing activities (to be captured in collected events) that will cause the subject to transition into a more desirable habit engagement state. As such, the predetermined period of time after which a subject has been associated with a particular habit engagement state that should prompt an intervention to be sent to the subject may be selected to be a period of time that is shorter than the determined drop off point corresponding to that state. In a second example, if a subject has recently transitioned to a particular habit engagement state, then that transition can be determined to motivate, guide, enable, prompt, and/or encourage a subject to perform a target activity.

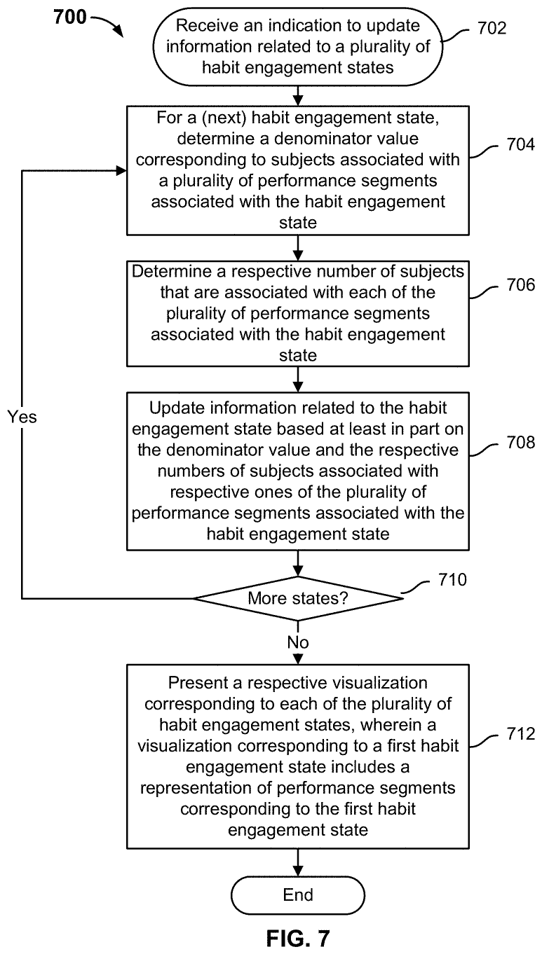

FIG. 7 is a flow diagram showing an example of a process for updating information related to a plurality of habit engagement states. In some embodiments, process 700 is implemented by habit engagement states tracking server 110 of system 100 of FIG. 1. In some embodiments, steps 302 and 304 of process 300 of FIG. 3 are implemented, at least in part, using process 700.

At 702, an indication to update information related to a plurality of habit engagement states is received. The indication to update information related to a plurality of habit engagement states may be received from a program or from a user instruction. For example, information related to a plurality of habit engagement states may be configured to be updated once an hour.

In some embodiments, the indication to update information related to a plurality of habit engagement states includes a set of filters that describe a subset of subjects for which the information related to the habit engagement states is to be updated and subsequently presented. For example, the filters may describe the age range, location, gender, and/or other attributes of subjects. If such filters were set, then only those subjects that match the criteria of the filters are to be considered in steps 704 through 712.

At 704, for a (next) habit engagement state, a denominator value corresponding to subjects associated with a plurality of performance segments associated with the habit engagement state is determined. For example, the denominator value corresponding to subjects associated with the plurality of performance segments is determined as the total number of subjects that have been previously associated with the habit engagement state or are currently associated with the habit engagement state. For example, the denominator value corresponding to subjects associated with the plurality of performance segments is determined as the total number of subjects that have been previously associated with the habit engagement state or are currently associated with a habit engagement state that is other than the current habit engagement state that is under consideration. For example, the total number of subjects that has been associated with the habit engagement state can be determined based on statistics that have been stored for the habit engagement state and/or performance segment memberships that have been stored for each subject.

At 706, a respective number of subjects that are associated with each of a plurality of performance segments associated with the habit engagement state is determined. For example, the number of subjects that is associated with each performance segment associated with the habit engagement state is determined based on the statistics that have been stored for the habit engagement state and/or performance segment memberships that have been stored for each subject. In some embodiments, there is at least one performance segment that is associated with each habit engagement state.

At 708, information related to the habit engagement state is updated based at least in part on the denominator value and the respective numbers of subjects associated with respective ones of the plurality of performance segments associated with the habit engagement state. In some embodiments, a corresponding percentage is determined for each performance segment of the habit engagement state as a function of the number of subjects associated with that performance segment divided by the denominator value corresponding to subjects associated with a plurality of performance segments associated with the habit engagement state. For example, assume that State A had two performance segments comprising a successful performance segment and an unsuccessful performance segment. Also, assume that State A has been and is currently associated with 100 total subjects of which 60 subjects belong to the successful performance segment and 40 subjects belong to the unsuccessful performance segment. The denominator value corresponding to subjects associated with the performance segments for State A is State A's 100 total subjects. As such, the information related to the habit engagement state would be 60% of subjects being associated with the successful performance segment and 40% of subjects being associated with the unsuccessful performance segment.

At 710, it is determined whether there is at least one more habit engagement state for which information has not yet been updated. In the event that there is at least one more habit engagement state for which information is to be updated, control is returned to 704. Otherwise, in the event that there are no more habit engagement states for which information is to be updated, control is transferred to 712.

At 712, a respective presentation corresponding to each of the plurality of habit engagement states is presented, wherein a presentation corresponding to a first habit engagement state includes a representation of performance segments corresponding to the first habit engagement state. A respective visualization of each habit engagement state of the plurality of habit engagement states is presented at a user interface. The presentation of each habit engagement state of the plurality of habit engagement states is sometimes referred to as a “dashboard.” A visualization of each habit engagement state can show representations associated with the state's performance segments. In some embodiments, the visualizations of the habit engagement state also show representations associated with the number, proportion, percentage, and/or other numeric value regarding the number of subjects that have historically transitioned from one habit engagement state to an adjacent habit engagement state. In some embodiments, the respective visualization of each habit engagement state also shows a confidence value associated with the confidence in the accuracy of the corresponding performance segment information. For example, the confidence in the accuracy of the corresponding performance segment information may be determined as a function of the source identifying information associated with the events that were used to generate the information. The respective visualization of each habit engagement state may include the presentation of additional or alternative types of information other than the examples described herein. In some embodiments, the presentation corresponding to each habit engagement state may also include statistics, text, as well as suggested interventions to be provided to subjects that current belong to that habit engagement state.

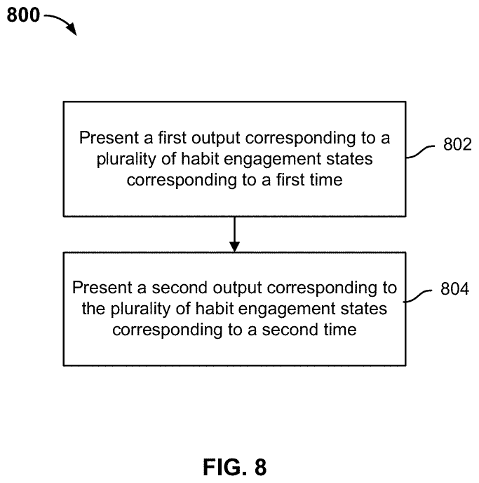

FIG. 8 is a flow diagram showing an example of a process for presenting two outputs corresponding to a plurality of habit engagement states. In some embodiments, process 800 is implemented by habit engagement states tracking server 110 of system 100 of FIG. 1.

Process 800 describes an example process by which the outputs of information pertaining to a plurality of habit engagement states that had been generated at different times are cached so that they can be presented in a manner that allows a habit dashboard user to see how subjects are progressing through the habit engagement states over time.

At 802, a first output corresponding to a plurality of habit engagement states corresponding to a first time is presented. For example, a first output (a dashboard presentation) corresponding to the habit engagement states was generated using a process such as process 700 of FIG. 7 at a first time and was cached.

At 804, a second output corresponding to the plurality of habit engagement states corresponding to a second time is presented. For example, a second output (a dashboard presentation) corresponding to the habit engagement states was generated using a process such as process 700 of FIG. 7 at a second time and was cached. For example, a habit dashboard user (e.g., a marketing professional) can switch between the dashboards corresponding to the first and second different times (and/or dashboards corresponding to a third or other time) to understand how subjects are progressing through the states over time. The habit dashboard user can use such presentations to determine whether any of the subjects need to be prompted or targeted via interventions to encourage them to perform one or more target activities that will lead to the formation of desired habits.

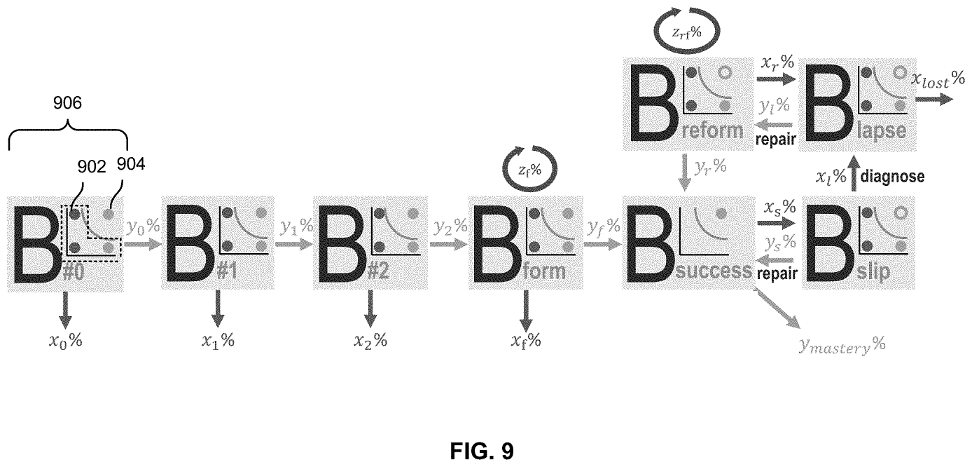

FIG. 9 is a diagram that shows an example output of a set of habit engagement states pertaining to forming the habit of regularly brushing teeth with an electronic toothbrush. The output of habit engagement states shown in FIG. 9 may be referred to as a dashboard of the habit engagement states corresponding a particular time. For example, the dashboard of the habit engagement states corresponding to a particular time refers to the output of habit engagement states that was determined based on events collected up until to the particular time. In some embodiments, the output of habit engagement states shown in FIG. 9 may be cached. In some embodiments, the output of habit engagement states shown in FIG. 9 may be generated using a process such as process 700 of FIG. 7. In the example of FIG. 9, the set of habit engagement states include States B_{#0}, B_{#1}, B_{#2}, B_{form}, B_{success}, Bₛₗᵢₚ, Bₗₐₚₛₑ, and B_{reform}. The states, which will be described in further detail below, describe stages that lead up to and away from the successful formation of the target habit of regularly brushing teeth with an electronic toothbrush. The events that are collected to generate and output the visualizations shown in FIG. 9 may be collected from the electronic toothbrushes, which are configured to detect and report the target activity of toothbrushing using the electronic toothbrush. As shown in FIG. 9, each state is shown with a corresponding visualization. For example, for State B_{#0}, visualization 906 shows the name of the State B_{#0} and a graph. The graph of visualization 906 corresponding to State B_{#0} includes three circles 902 to the left of the plotted curve and one circle 904 to the right of the plotted curve. In the specific example of FIG. 9, three circles 902 collectively represent the portion of subjects that have been associated with State B_{#0} and are part of the unsuccessful performance segment of State B_{#0} and circle 904 represent the portion of subjects that have been associated with State B_{#0} and are part of the successful performance segment of State B_{#0}.

As shown by the arrows with corresponding x or y values in FIG. 9, a state for which either or both x or y arrows points towards the state's visualization is considered to be an adjacent state relative to the state of the visualization from which the arrows point away. When a subject's associated events that describe activities meet the transition logic from the subject's current state to a state that is adjacent to the subject's current state, then that subject is determined to transition to be associated with the adjacent state. For example, State B_{#1} is an adjacent state to State B_{#0}; State B_{#2} is an adjacent state to State B_{#1}; State B_{form} is an adjacent state to State B_{#2}; State B_{success} is an adjacent state to States B_{form}, Bₛₗᵢₚ, and B_{reform}; State Bₛₗᵢₚ is adjacent to State B_{success}; State Bₗₐₚₛₑ is adjacent to both States B_{reform} and Bₛₗᵢₚ; and B_{reform} is adjacent to State Bₗₐₚₛₑ. In the specific example of FIG. 9, each of the x, y, and z values that are shown relative to the visualization of a state respectively represents the respective percentage of subjects that are associated with that state and that belong to a respective performance segment associated with that state.

In the specific example of FIG. 9, either x or y values with corresponding arrows that point from a first state's visualization towards a second state's visualization represents not only the respective percentage of subjects that are associated with the first state and belong to a respective performance segment associated with the first state, but also the percentage of subjects that have transitioned from the first state to the second state. This is a result of the transition logic of the first state to the second state matching at least a portion of the segmenting logic of the first state. For example, y_{0%} represents both the percentage of subjects that had been associated with the successful performance segment of State B_{#0} and that have also met the transition criteria to transition to being associated with State B_{#1}.

The following is an example set of configuration information for the states shown in FIG. 9:

State B_{#0}: The onboarding of the electronic toothbrush stage

Segmenting logic for State B_{#0}:

The successful performance segment includes those subjects who have completed onboarding (e.g., downloading an application to be used with the electronic toothbrushes and/or performing electronic registration) for their electronic toothbrushes. The percentage (y_{0%}) of subjects associated with the successful performance segment is determined as the number of subjects who have completed onboarding divided by the total number of subjects who have received an electronic toothbrush.

The unsuccessful performance segment includes those subjects who have not completed onboarding for their electronic toothbrushes. The percentage (x_{0%}) of subjects associated with the unsuccessful performance segment is determined as the number of subjects who are not known to have completed onboarding divided by the total number of subjects who have received an electronic toothbrush.

Transition logic from State B_{#0} to B_{#1}: Those subjects who have completed onboarding for their electronic toothbrushes. The percentage of subjects that transition from State B_{#0} to B_{#1} is represented by y_{0%}.

State B_{#1}: The completion of the target activity (e.g., using the electronic toothbrush) for the first time stage

Segmenting logic for State B_{#1}:

The successful performance segment includes those subjects who have completed the target activity (e.g., using the electronic toothbrush) once. The percentage (y_{1%}) of subjects associated with the successful performance segment is determined as the number of subjects who have completed the target activity once divided by the total number of subjects who have transitioned from State B_{#0} and became associated with State B_{#1}.

The unsuccessful performance segment includes those subjects who have not completed the target activity once. The percentage (x_{1%}) of subjects associated with the unsuccessful performance segment is determined as the number of subjects who have not completed the target activity once divided by the total number of subjects who have transitioned from State B_{#0} and became associated with State B_{#1}.

Transition logic from State B_{#1} to B_{#2}: Those subjects who have completed the target activity once. The percentage of subjects that transition from State B_{#1} to B_{#2} is represented by y_{1%}.

State B_{#2}: The completion of the target activity for a second time stage

Segmenting logic for State B_{#2}:

The successful performance segment includes those subjects who have completed the target activity (e.g., using the electronic toothbrush) twice on two different days (e.g., the subject completed the target activity once on one day and then the subject completed the target activity a second time on another day). The percentage (y_{2%}) of subjects associated with the successful performance segment is determined as the number of subjects who have completed the target activity twice on two different days divided by the total number of subjects who have transitioned from State B_{#1} and became associated with State B_{#2}.

The unsuccessful performance segment includes those subjects who have not completed the target activity twice on two different days. The percentage (x_{2%}) of subjects associated with the unsuccessful performance segment is determined as the number of subjects who have not completed the target activity twice on two different days divided by the total number of subjects who transitioned from State B_{#1} and became associated with State B_{#2}.

Transition logic from State B_{#2} to B_{form}: Those subjects who have completed the target activity twice on two different days. The percentage of subjects that transition from State B_{#2} to B_{form} is represented by y_{2%}.

State B_{form}: The stage where subjects are forming the target habit but have not yet met the criteria for the B_{success}

The in-habit formation performance segment includes those subjects who are in the process of forming the target habit by having completed the target activity (e.g., using the electronic toothbrush) more than 21 times but fewer than 24 times in the last 30 days. The percentage (z_{f%}) of subjects associated with the in-habit formation performance segment is determined as the number of subjects who have completed the target activity more than 21 times but fewer than 24 times in the last 30 days divided by the total number of subjects who have transitioned from State B_{#1} and became associated with State B_{#2}.

The successful performance segment includes those subjects who are actively forming the target habit by having completed the target habit 28 or more times in the last 30 days. The percentage (y_{f%}) of subjects associated with the successful performance segment is determined as the number subjects who have completed the target habit 28 or more times in the last 30 days divided by the total number of subjects who transitioned from State B_{#1} and became associated with State B_{#2}.

The unsuccessful performance segment includes those subjects who are not actively forming the target activity by only having completed the target activity for 20 or fewer days out of the last 30 days. The percentage (x_{f%}) of subjects associated with the unsuccessful performance segment is determined as the number subjects who have only completed the target activity for 20 or fewer days out of the last 30 days divided by the total number of subjects who have transitioned from State B_{#1} and became associated with State B_{#2}.

Transition logic from State B_{form} to B_{success}: Those subjects who have completed the target habit 28 or more times in the last 30 days. The percentage of subjects that transition from State B_{form} to B_{success} is represented by y_{f%}.

State B_{success}: The stage where subjects have successfully formed the target habit

The successful performance segment includes those subjects who continue to complete the target activity 28 or more times in the last 30 days. The percentage (y_{mastery%}) of subjects associated with the successful performance segment is determined as the number subjects who continue to complete the target habit 28 or more times in the last 30 days divided by the total number of subjects who transitioned from State B_{form} and became associated with State B_{success}. For example, the y_{mastery%} percentage of subjects no longer transition to another state.

The unsuccessful performance segment includes those subjects who have missed the target activity for 12 days in a row. The percentage (x_{s%}) of subjects associated with the unsuccessful performance segment is determined as the number of subjects who have missed the target activity for 12 days in a row divided by the total number of subjects who have transitioned from State B_{form} and became associated with State B_{success}.

Transition logic from State B_{success}to Bₛₗᵢₚ: Those subjects who missed the target activity for 12 days in a row. The percentage of subjects that transition from State B_{success}to Bₛₗᵢₚ is represented by x_{s%}.

State Bₛₗᵢₚ: The stage where subjects have briefly failed to keep up with the target habit but might recover.

The successful performance segment includes those subjects who missed completing the target activity 6 times but then performed the target activity successfully on the 7th day. The percentage (y_{s%}) of subjects associated with the successful performance segment is determined as the number subjects who missed completing the target activity only 6 times but then performed the target activity successfully on the 7th day divided by the total number of subjects who transitioned from State B_{form} and became associated with State B_{success}.

The unsuccessful performance segment includes those subjects who have missed the target activity for 21 days or more in a row. The percentage (x_{1%}) of subjects associated with the unsuccessful performance segment is determined as the number subjects who have missed the target activity for 21 days or more in a row divided by the total number of subjects who have transitioned from State B_{form} and became associated with State B_{success}.

Transition logic from State Bₛₗᵢₚ to Bₗₐₚₛₑ: Those subjects who have missed the target activity for 21 days or more in a row. The percentage of subjects that transition from State Bₛₗᵢₚ to Bₗₐₚₛₑ is represented by x_{1%}.

Transition logic from State Bₛₗᵢₚ to B_{success}: Those subjects who missed completing the target activity only 6 times but then performed the habit successfully on the 7th day. The percentage of subjects that transition from State Bₛₗᵢₚ to B_{success} is represented by y_{s%}.

State Bₗₐₚₛₑ: The stage where subjects have failed to keep up with the target activity and are not yet reforming the target habit.

The successful performance segment includes those subjects who have completed the target activity on 12 days in the last 30 days. The percentage (y_{1%}) of subjects associated with the successful performance segment is determined as the number of subjects who have completed the target activity on 12 days in the last 30 days divided by the denominator value comprising the total number of subjects who transitioned from State B_{form} and became associated with State B_{success}.

The unsuccessful performance segment includes those subjects who have not completed the target activity for 12 days in the last 30 days. The percentage (x_{lost%}) of subjects associated with the unsuccessful performance segment is determined as the number of subjects who have not completed the target activity for 12 days in the last 30 days divided by the denominator value comprising the total number of subjects who have transitioned from State B_{form} and became associated with State B_{success}.

Transition logic from State Bₗₐₚₛₑ to B_{reform}: Those subjects who have completed the target activity on at least 12 days in the last 30 days. The percentage of subjects that transition from State Bₗₐₚₛₑ to B_{reform} is represented by y_{1%}.

State B_{reform}: The stage where subjects are reforming the target habit after failing to keep up with the target activity

The in-habit reformation performance segment includes those subjects who have completed the target activity more than 21 times but fewer than 24 times in the last 30 days. The percentage (z_{rf%}) of subjects associated with the in-habit reformation performance segment is determined as the number subjects who have completed the target activity more than 21 times but fewer than 24 times in the last 30 days divided by the denominator value the total number of subjects who have transitioned from State B_{form} and became associated with State B_{success}.

The successful performance segment includes those subjects who are actively re-forming the target habit by having completed the target activity 28 or more times in the last 30 days. The percentage (y_{r%}) of subjects associated with the successful performance segment is determined as the number subjects who have completed the target habit 28 or more times in the last 30 days divided by the denominator value the total number of subjects who transitioned from State B_{form} and became associated with State B_{success}.

The unsuccessful performance segment includes those subjects who are not actively re-reforming the target habit by having completed the target behavior on only 1 day out of the last 30 days. The percentage (x_{r%}) of subjects associated with the unsuccessful performance segment is determined as the number subjects who have completed the target activity on only 1 days out of the last 30 days divided by the denominator value the total number of subjects who have transitioned from State B_{form} and became associated with State B_{success}.

Transition logic from State B_{reform} to Bₗₐₚₛₑ: Those subjects who have completed the target behavior on only 1 day out of the last 30 days. The percentage of subjects that transition from State B_{reform} to Bₗₐₚₛₑ is represented by x_{r%}.

Transition logic from State B_{reform} to B_{success}: Those subjects who have completed the target activity 28 or more times in the last 30 days. The percentage of subjects that transition from State B_{reform} to B_{success}is also represented by y_{r%}.

The dashboard output of FIG. 9 can provide the viewing habit dashboard user insight on how subjects that are performing target activities (toothbrushing with the electronic toothbrush) lead up to forming habits (regularly toothbrushing with the electronic toothbrush) such as by what percentage of subjects transitions from one state to the next. The dashboard output can enable the viewing habit dashboard user to determine at what stage of the habit engagement process subjects encounter difficulties and seem less likely to advance to the next stage towards successfully forming a habit. In response to that determination, the habit dashboard user and/or a system driven by artificial intelligence (e.g., machine learning) can determine how best to design and deliver an intervention that will help the subjects perform the target activities.

In some embodiments, the representation associated with each performance segment corresponding to a state (e.g., circle 904 associated with the successful performance segment of the visualization of State B_{#0}) is an interactable element that a habit dashboard user can select. In response to selecting such a representation that is associated with each performance segment corresponding to a state, a corresponding user interface is displayed that shows a user interface that presents additional information corresponding to the performance segment. For example, the user interface that presents additional information corresponding to the performance segment may include confidence levels related to the data represented in that performance segment, a description of the subjects that are included in that performance segment, any issues that the subjects that are included in that performance segment may have with respect to forming the target habit, and/or any recommended actions/prompts/interventions to perform to encourage the subjects to form the target habit.

In some embodiments, the representation associated with each performance segment corresponding to a state (e.g., circle 904 associated with the successful performance segment of the visualization of State B_{#0}) may be associated with a size, for example, that is relative to the number of the subjects that are included in the performance segment. For example, the more subjects that are included in the performance segment, the larger the representation is of the performance segment.

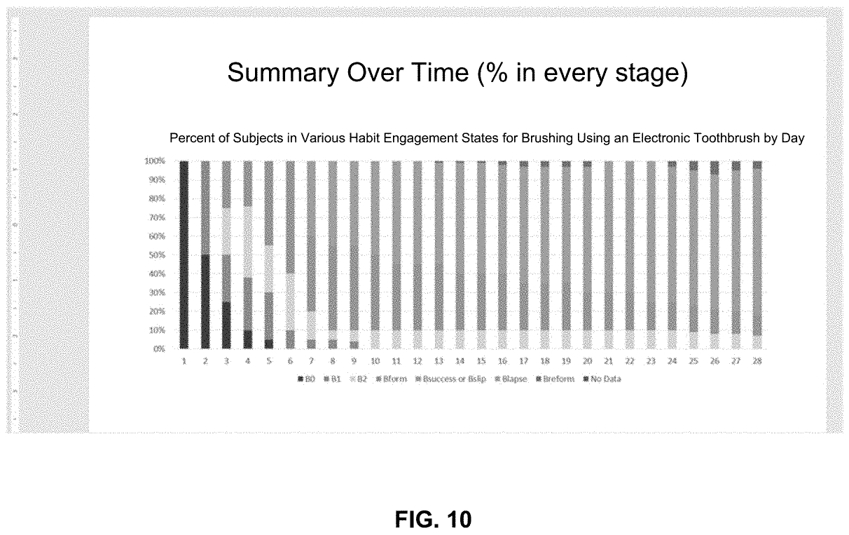

FIG. 10 is a diagram showing an example user interface for presenting detailed information associated with a set of habit engagement states. As mentioned above, in some embodiments, the output of performance segment information associated with a set of habit engagement states is an interactable presentation, such as the dashboard presentation that is presented in FIG. 9. In response to a user selection on a portion of the dashboard presentation, in some embodiments, additional, detailed information associated with a set of habit engagement states may be presented. The plot that is shown in FIG. 10 is one example of additional, detailed information associated with a set of habit engagement states that can be presented in response to a user selection (e.g., a click) on a dashboard presentation of performance segment information related to a set of habit engagement states. Specifically, the example plot in FIG. 10 shows the percentage breakdown of the tracked population of subjects in respective habit engagement states that are described with FIG. 9 per day over 28 days.

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. A system, comprising:
a processor configured to:
obtain events related to a plurality of subjects from a plurality of networked appliances, wherein the plurality of networked appliances includes sensors that detect the events;
determine performance segment information corresponding to respective ones of a plurality of habit engagement states based at least in part on the events; and
output at least a subset of the performance segment information corresponding to the respective ones of the plurality of habit engagement states, wherein the output comprises a representation of a portion of subjects that have transitioned between two adjacent habit engagement states; and
a memory coupled to the processor and configured to provide the processor with instructions.

2. The system of claim 1, wherein the processor is further configured to:
present a user interface;
obtain, via the user interface, identifying information associated with the plurality of habit engagement states;
obtain, via the user interface, segmenting logic associated with the plurality of habit engagement states; and
obtain, via the user interface, transition logic associated with the plurality of habit engagement states.

3. The system of claim 1, wherein the processor is configured to:
obtain a new event;
determine a subject associated with the new event;
determine a stored current habit engagement state associated with the subject;
determine that the new event was received over a predetermined length of time after a previous event associated with the subject was received; and
in response to the determination that the new event was received over the predetermined length of time after a previous event associated with the subject was received, update the stored current habit engagement state associated with the subject to a predetermined habit engagement state.

4. The system of claim 1, wherein the processor is configured to:
obtain a new event;
determine a subject associated with the new event;
determine a stored current habit engagement state associated with the subject; and
determine and update performance segment statistics corresponding to the stored current habit engagement state.

5. The system of claim 1, wherein the processor is configured to:
obtain a new event;
determine a subject associated with the new event;
determine a stored current habit engagement state associated with the subject;
determine that the subject has transitioned to an adjacent habit engagement state;
in response to the determination that the subject has transitioned to the adjacent habit engagement state, update the stored current habit engagement state to the adjacent habit engagement state; and
update a set of historical events associated with the subject with the new event.

6. The system of claim 5, wherein the determination that the subject has transitioned to the adjacent habit engagement state is based at least in part on the new event, the set of historical events associated with the subject, and transition logic associated with the plurality of habit engagement states.

7. The system of claim 1, wherein to determine the performance segment information corresponding to the respective ones of the plurality of habit engagement states comprises to:
determine a denominator value corresponding to subjects associated with a plurality of performance segments associated with a first habit engagement state;
determine a respective number of subjects that is associated with each of the plurality of performance segments associated with the first habit engagement state; and
update a portion of the performance segment information related to the first habit engagement state based at least in part on the denominator value and the respective numbers of subjects associated with respective ones of the plurality of performance segments associated with the first habit engagement state.

8. The system of claim 1, wherein to output the at least subset of the performance segment information corresponding to the respective ones of the plurality of habit engagement states comprises to output respective visualizations corresponding to the plurality of habit engagement states, wherein a first visualization corresponding to a first habit engagement state includes representation of performance segments corresponding to the first habit engagement state.

9. (canceled)

10. The system of claim 1, wherein to output the at least subset of the performance segment information corresponding to the respective ones of the plurality of habit engagement states comprises to:
present a first output corresponding to the plurality of habit engagement states corresponding to a first time; and
present a second output corresponding to the plurality of habit engagement states corresponding to a second time.

11. A method, comprising:
obtaining events related to a plurality of subjects from a plurality of networked appliances, wherein the plurality of networked appliances includes sensors that detect the events;
determining performance segment information corresponding to respective ones of a plurality of habit engagement states based at least in part on the events; and
outputting at least a subset of the performance segment information corresponding to the respective ones of the plurality of habit engagement states, wherein the output comprises a representation of a portion of subjects that have transitioned between two adjacent habit engagement states.

12. The method of claim 11, further comprising:
presenting a user interface;
obtaining, via the user interface, identifying information associated with the plurality of habit engagement states;
obtaining, via the user interface, segmenting logic associated with the plurality of habit engagement states; and
obtaining, via the user interface, transition logic associated with the plurality of habit engagement states.

13. The method of claim 11, further comprising:
obtaining a new event;
determining a subject associated with the new event;
determining a stored current habit engagement state associated with the subject;
determining that the new event was received over a predetermined length of time after a previous event associated with the subject was received; and
in response to the determination that the new event was received over the predetermined length of time after a previous event associated with the subject was received, updating the stored current habit engagement state associated with the subject to a predetermined habit engagement state.

14. The method of claim 11, further comprising:
obtaining a new event;
determining a subject associated with the new event;
determining a stored current habit engagement state associated with the subject; and
determining and update performance segment statistics corresponding to the stored current habit engagement state.

15. The method of claim 11, further comprising:
obtaining a new event;
determining a subject associated with the new event;
determining a stored current habit engagement state associated with the subject;
determining that the subject has transitioned to an adjacent habit engagement state;
in response to the determination that the subject has transitioned to the adjacent habit engagement state, updating the stored current habit engagement state to the adjacent habit engagement state; and
update a set of historical events associated with the subject with the new event.

16. The method of claim 15, wherein the determination that the subject has transitioned to the adjacent habit engagement state is based at least in part on the new event, the set of historical events associated with the subject, and transition logic associated with the plurality of habit engagement states.

17. The method of claim 11, wherein determining the performance segment information corresponding to the respective ones of the plurality of habit engagement states comprises:
determining a denominator value corresponding to subjects associated with a plurality of performance segments associated with a first habit engagement state;
determining a respective number of subjects that is associated with each of the plurality of performance segments associated with the first habit engagement state; and
updating a portion of the performance segment information related to the first habit engagement state based at least in part on the denominator value and the respective numbers of subjects associated with respective ones of the plurality of performance segments associated with the first habit engagement state.

18. The method of claim 11, wherein outputting the at least subset of the performance segment information corresponding to the respective ones of the plurality of habit engagement states comprises to output respective visualizations corresponding to the plurality of habit engagement states, wherein a first visualization corresponding to a first habit engagement state includes representation of performance segments corresponding to the first habit engagement state.

19. (canceled)

20. A computer program product, the computer program product being embodied in a non-transitory computer readable storage medium and comprising computer instructions for:
obtaining events related to a plurality of subjects from a plurality of networked appliances, wherein the plurality of networked appliances includes sensors that detect the events;
determining performance segment information corresponding to respective ones of a plurality of habit engagement states based at least in part on the events; and
outputting at least a subset of the performance segment information corresponding to the respective ones of the plurality of habit engagement states, wherein the output comprises a representation of a portion of subjects that have transitioned between two adjacent habit engagement states.
